(19) United States
(12) Patent Application Publication
(10) Pub. No.: US 2011/0227052 A1
(43) Pub. Date: Sep. 22, 2011

(54) NOVEL ORGANIC COMPOUND, LIGHT-EMITTING DEVICE, AND IMAGE DISPLAY APPARATUS

(75) Inventors: Jun Kamatani, Tokyo (JP); Naoki Yamada, Inagi-shi (JP); Akihito Saitoh, Yokohama-shi (JP)

(73) Assignee: CANON KABUSHIKI KAISHA, Tokyo (JP)

(21) Appl. No.: 13/129,740

(22) PCT Filed: Nov. 17, 2009
(86) PCT No.: PCT/JP2009/069756

§ 371 Date: May 17, 2011

(30) Foreign Application Priority Data
Nov. 19, 2008 (JP) 2008-295801

(51) Int. Cl. H01L 51/54; C07C 13/62

(52) U.S. Cl. 257/40; 585/27; 257/E51.024

(57) ABSTRACT

An organic compound represented by general formula (1) below and an organic light-emitting device including the organic compound are provided.

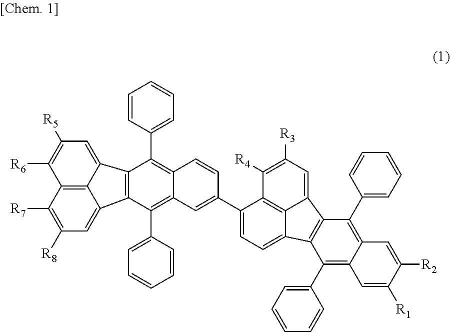

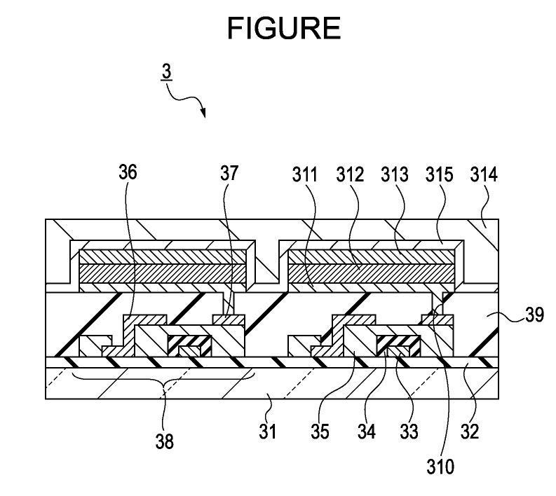

### TECHNICAL FIELD

The present invention relates to a light-emitting device including an organic compound, and an organic light-emitting device (also referred to as “organic electroluminescence device” or “organic EL device”) used in a surface light source, a planar display, or the like.

### BACKGROUND ART

Organic light-emitting devices include an anode, cathode, and a thin film containing a fluorescent organic compound disposed between an anode and a cathode. Excitons of the fluorescent compound are generated by injecting electrons and holes from the electrodes, and the organic light-emitting devices utilize light emitted when the excitons are returned to the ground state.

Recently, organic light-emitting devices have become markedly advanced and the possibility of a wide variety of applications has been suggested therefor because of the high luminance achieved by a low applied voltage, a variety of emission wavelengths, a high-speed responsiveness, and the possibility of realization of a thin, lightweight light-emitting device.

However, under the present situation, an optical output with a higher luminance and higher conversion efficiency are necessary. Furthermore, there are still a lot of problems in terms of durability, for example, a change with time due to long-term use and degradation due to an atmospheric gas containing oxygen, moisture, or the like.

Furthermore, considering an application to a full-color display or the like, a blue-light emission having good color purity and a high luminous efficiency is necessary, but technologies related to these issues have not yet satisfactorily been developed. In addition, in particular, an organic light-emitting device having high color purity, luminous efficiency, and durability and a material that realizes such an organic light-emitting device have been desired. Patent Citations 1 to 5 describe that an organic compound having a 7,12-diphenylbenzo[k]fluoranthene skeleton is used in a light-emitting device.

### Patent Citation 1

Japanese Patent Laid-Open No. 10-189247

### Patent Citation 2

Japanese Patent Laid-Open No. 2005-235787

### Patent Citation 3

Japanese Patent Laid-Open No. 2003-026616

### Patent Citation 4

PCT Publication No. WO2008-015945

### Patent Citation 5

PCT Publication No. WO2008-059713

### DISCLOSURE OF INVENTION

The present invention has been made to solve the above-described problems in the related art. The present invention provides an organic light-emitting device that includes an organic compound suitable for blue-light emission and that emits light with a high efficiency and a high luminance. Furthermore, the present invention provides a durable organic light-emitting device.

The present invention provides an organic compound represented by general formula (I) below.

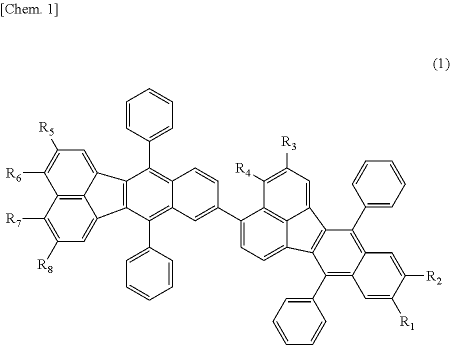

In general formula (I), R₁ to R₈ are each independently selected from a hydrogen atom, a halogen atom, a substituted or unsubstituted alkyl group, a substituted or unsubstituted alkoxy group, a substituted or unsubstituted aralkyl group, a substituted or unsubstituted amino group, a substituted or unsubstituted aryl group, and a substituted or unsubstituted heterocyclic group.

Furthermore, the present invention provides an organic light-emitting device including a cathode, an anode, and an organic compound layer disposed between the anode and the cathode, wherein the organic compound layer contains an organic compound in which two 7,12-diphenylbenzo[k]fluoranthene skeletons each of which may have a substituent are bonded head-to-tail or bonded tail-to-tail at positions different from each other.

An organic light-emitting device including the organic compound of the present invention can realize light emission with a high efficiency and a high luminance. In addition, a durable organic light-emitting device can be realized.

### BRIEF DESCRIPTION OF DRAWING

FIGURE is a schematic cross-sectional view showing organic light-emitting devices and TFTs provided under the organic light-emitting devices.

### DESCRIPTION OF EMBODIMENTS

An organic compound according to the present invention is represented by general formula (1) below.

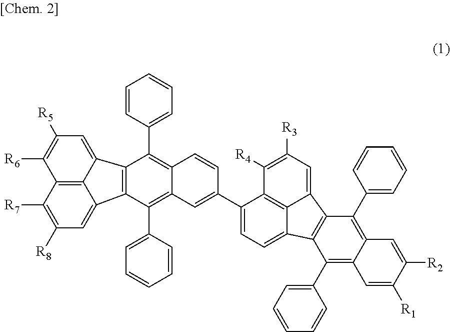

In general formula (I), R₁ to R₈ are each independently selected from a hydrogen atom, a halogen atom, a substituted or unsubstituted alkyl group, a substituted or unsubstituted alkoxy group, a substituted or unsubstituted aralkyl group, a substituted or unsubstituted amino group, a substituted or unsubstituted aryl group, and a substituted or unsubstituted heterocyclic group.

Specific examples of the substituents, i.e., the halogen atom, the alkyl group, the alkoxy group, the aralkyl group, the amino group, the aryl group, and the heterocyclic group of the compound represented by general formula (I) will be described below.

Examples of the halogen atom include atoms of fluorine, chlorine, bromine, and iodine.

Examples of the alkyl group include, but are not limited to, a methyl group, an ethyl group, a n-propyl group, an isopropyl group, a n-butyl group, a tert-butyl group, a sec-butyl group, an octyl group, a 1-adamantyl group, and a 2-adamantyl group.

Examples of the alkoxy group include, but are not limited to, a methoxy group, an ethoxy group, a propoxy group, a 2-ethyl-octyloxy group, a phenoxy group, a 4-tert-butylphenoxy group, a benzyloxy group, and a thienyloxy group.

Examples of the aralkyl group include, but are not limited to, a benzyl group.

Examples of the substituted amino group include, but are not limited to, an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-tert-butylphenyl)amino group, and an N-phenyl-N-(4-trifluoromethylphenyl)amino group.

Examples of the aryl group include, but are not limited to, a phenyl group, a naphthyl group, an indenyl group, a biphenyl group, a terphenyl group, and a fluorenyl group.

Examples of the heterocyclic group include, but are not limited to, a pyridyl group, an oxazolyl group, an oxadiazolyl group, a thiazolyl group, a thiadiazolyl group, a carbazolyl group, an acridinyl group, and a phenanthrolyl group.

The above-mentioned substituents may have a substituent. Examples of the substituent include alkyl groups such as a methyl group, an ethyl group, and a propyl group; aralkyl groups such as a benzyl group; aryl groups such as a phenyl group and a biphenyl group; heterocyclic groups such as a pyridyl group and a pyrrolyl group; amino groups such as a dimethylamino group, a diethylamino group, a dibenzylamino group, a diphenylamino group, and a ditolylamino group; alkoxyl groups such as a methoxyl group, an ethoxyl group, a propoxyl group, and a phenoxyl group; a cyano group; and halogen atoms such as fluorine, chlorine, bromine, and iodine.

The organic compound represented by general formula (I) is an organic compound in which two 7,12-diphenylbenzo[k]fluoranthene skeletons are bonded head-to-tail at positions different from each other.

In general formula (I), the 9-position of a 7,12-diphenylbenzo[k]fluoranthene skeleton is bonded to the 3-position of another 7,12-diphenylbenzo[k]fluoranthene skeleton.

In the present invention, the head of a 7,12-diphenylbenzo[k]fluoranthene skeleton means the 9-position and the 10-positions of the 7,12-diphenylbenzo[k]fluoranthene skeleton. At this head, i.e., at least one of the 9-position and the 10-position, this 7,12-diphenylbenzo[k]fluoranthene skeleton is bonded to another 7,12-diphenylbenzo[k]fluoranthene skeleton.

The tail of a 7,12-diphenylbenzo[k]fluoranthene skeleton means the 2-position to the 5-position of the 7,12-diphenylbenzo[k]fluoranthene skeleton.

At this tail, i.e., at least one of the 2-position to the 5-position, this 7,12-diphenylbenzo[k]fluoranthene skeleton is bonded to another 7,12-diphenylbenzo[k]fluoranthene skeleton.

The inventors of the present invention found that such organic compounds represented by general formula (I) are useful for an organic light-emitting device.

An organic light-emitting device includes a pair of electrodes, i.e., an anode and a cathode, and an organic compound layer disposed between the electrodes.

The inventors of the present invention believe that organic compounds other than the organic compounds represented by general formula (I) are also useful for such an organic light-emitting device.

Specifically, it is believed that organic compounds in which two 7,12-diphenylbenzo[k]fluoranthene skeletons are bonded head-to-tail or bonded tail-to-tail at positions different from each other are also useful.

The following compounds are exemplified as organic compounds that are preferably used in an organic light-emitting device. In particular, Exemplified Compounds A1 to A19 are included in the compounds represented by general formula (I) above.

A group of B1 to B9 and a group of B10 to B15 are cited as organic compounds which are believed to be preferably used in an organic light-emitting device.

The present invention is not limited to exemplified compounds below.

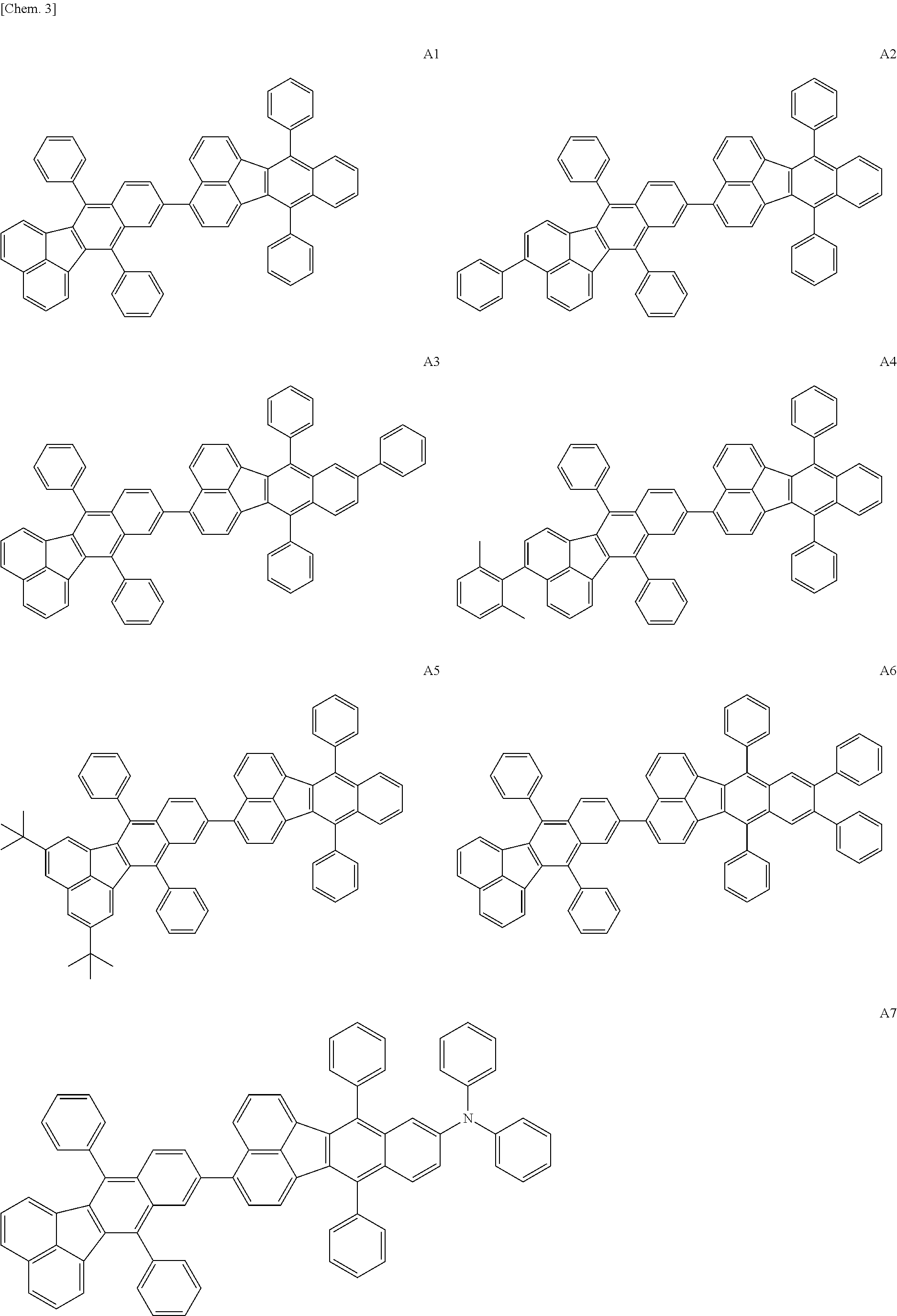

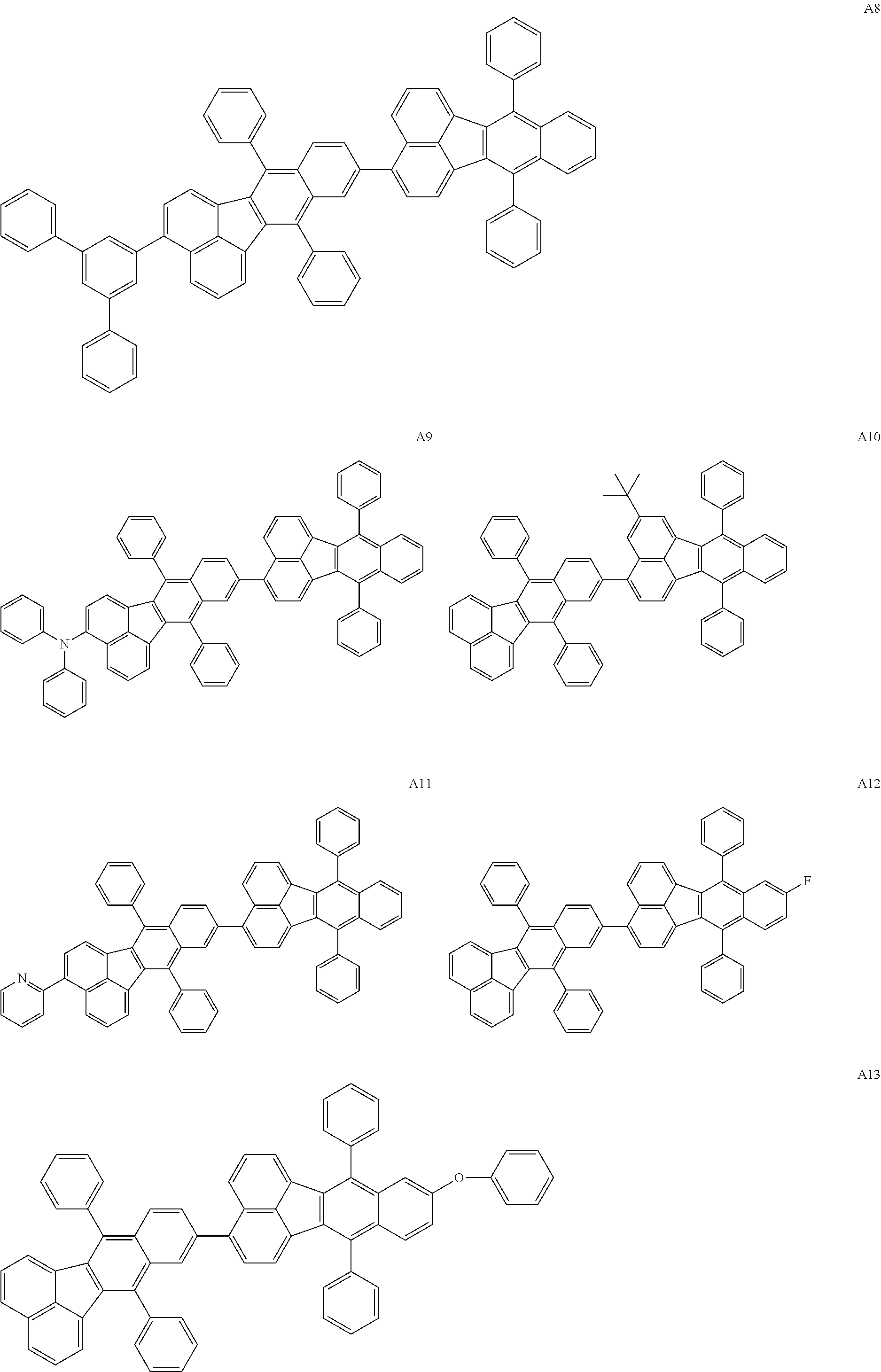

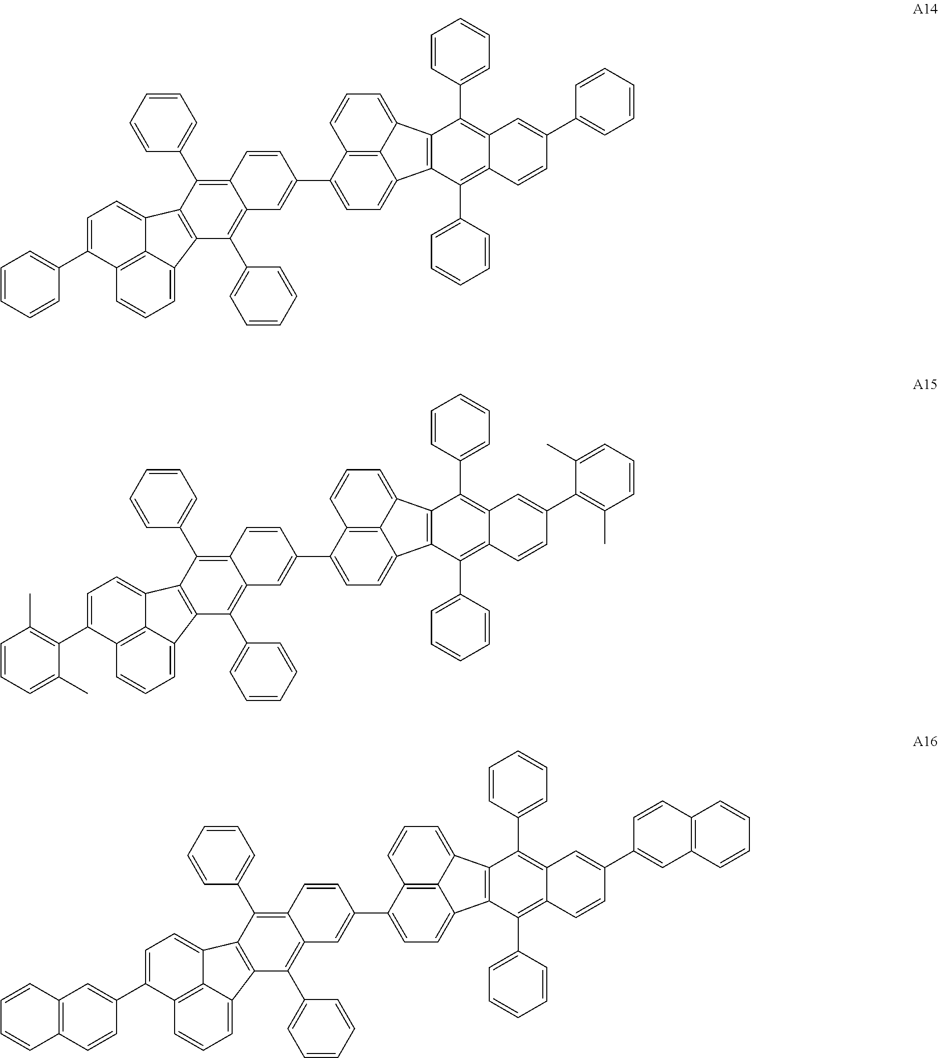

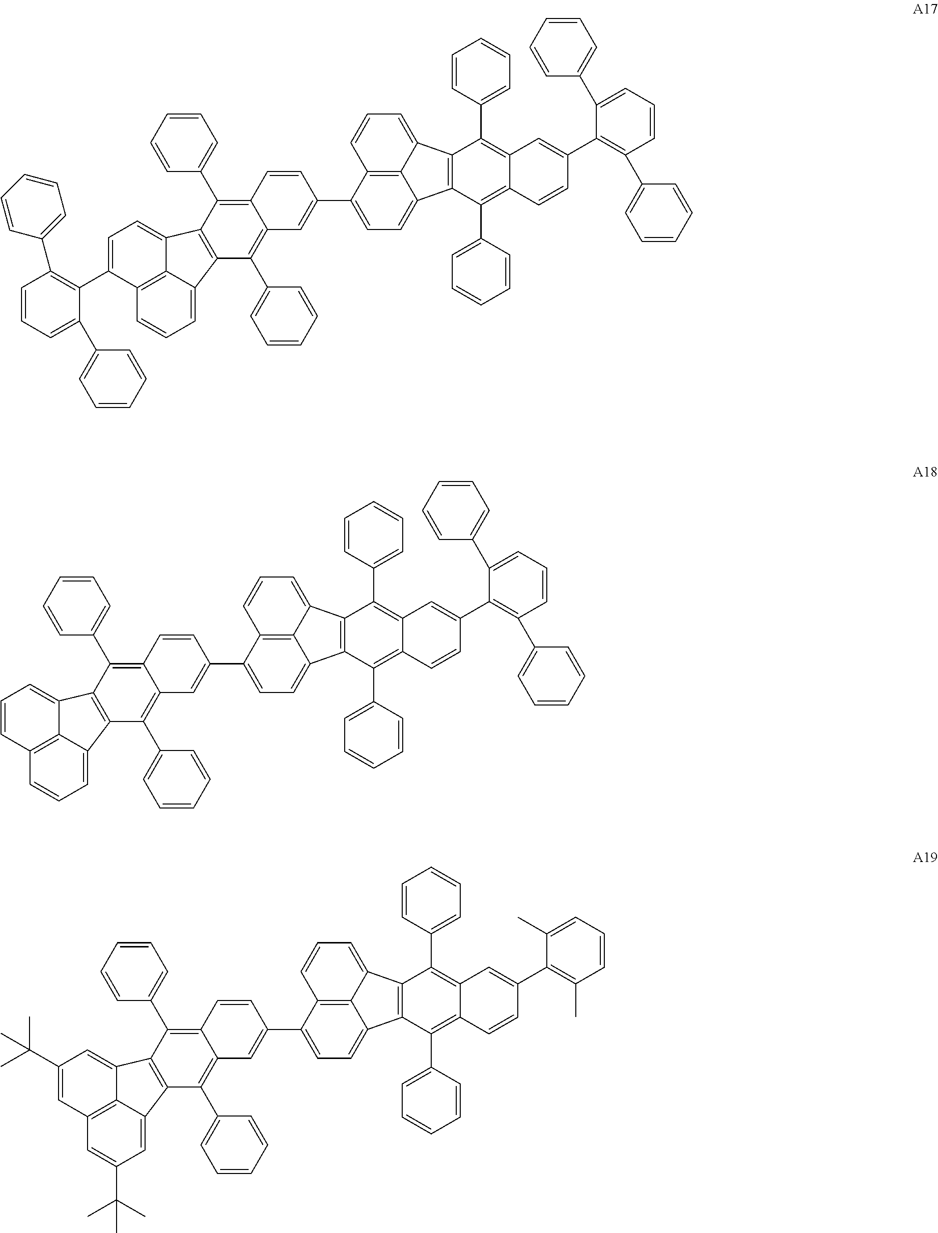

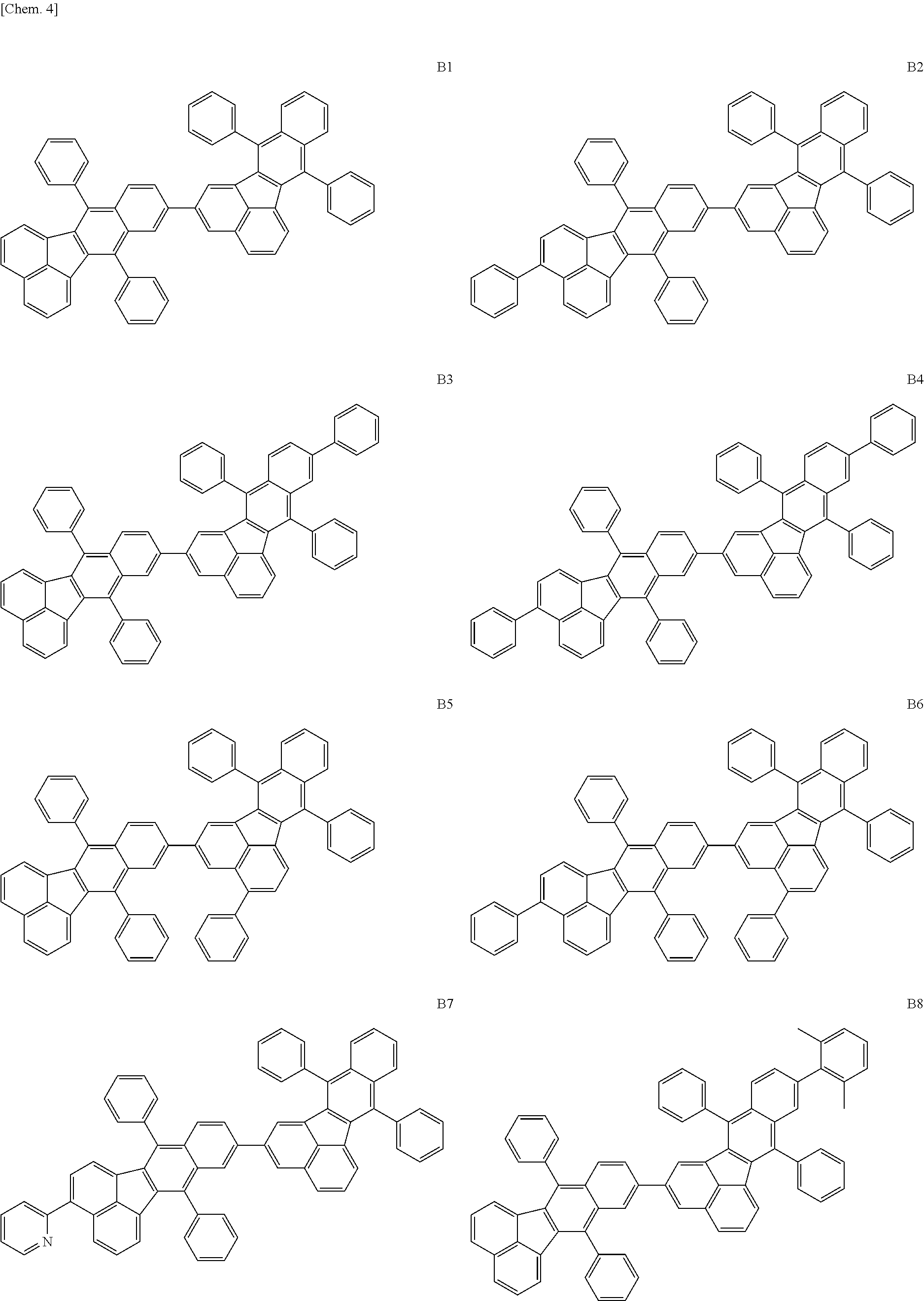

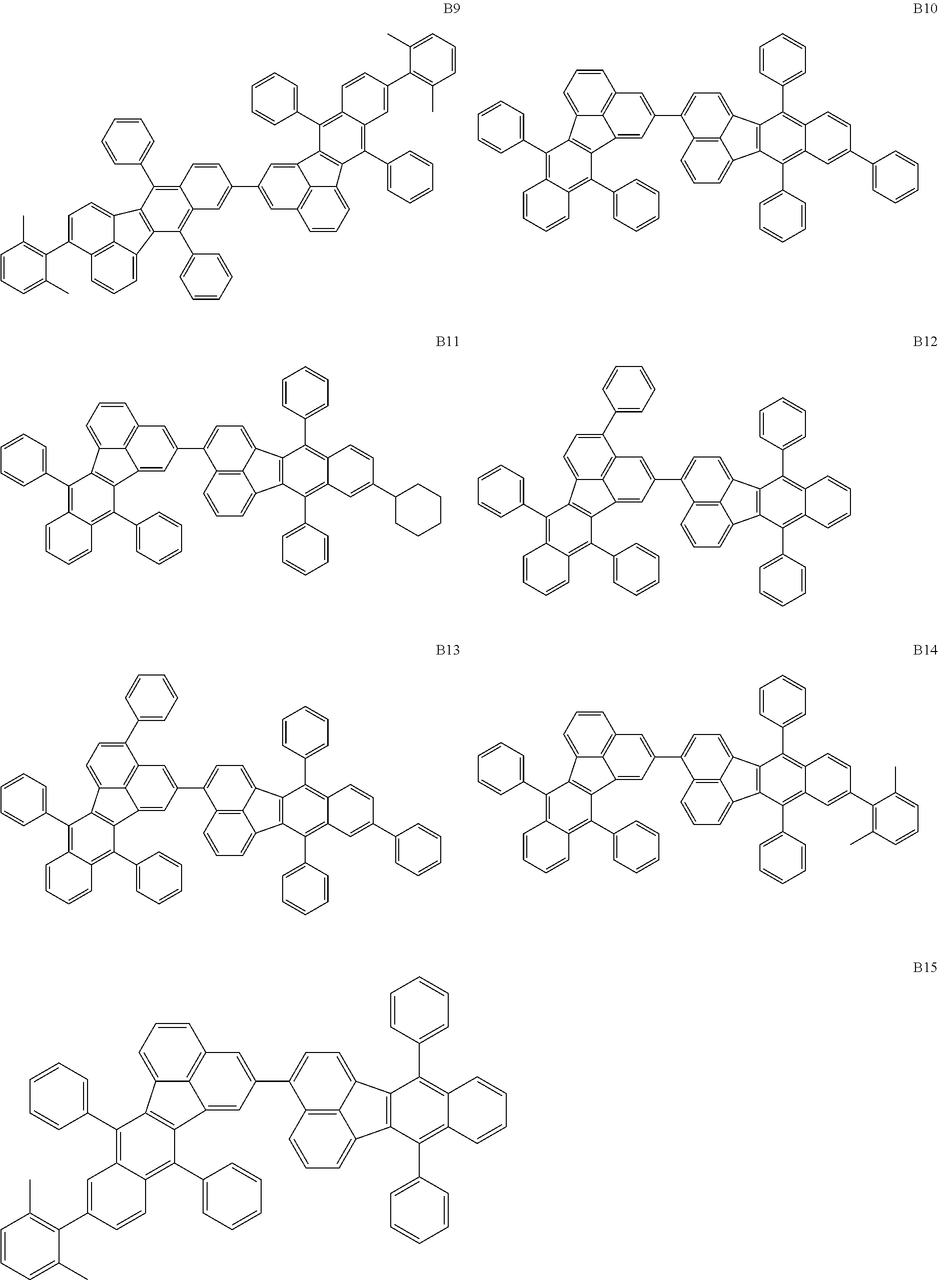

Organic compounds according to the present invention will be described in more detail below.

In general, in order to increase the luminous efficiency of an organic light-emitting device, it is desirable that the emission quantum yield of a luminescence center material is high.

As a result of studies made by the inventors of the present invention, it was found that organic compounds represented by general formula (I) have a high quantum yield in a dilute solution. Therefore, when organic compounds represented by general formula (I) are used in an organic light-emitting device, a high luminous efficiency can be expected.

The organic compound of the present invention may have a fluoranthenyl group at the 9-position of a 7,12-diphenylbenzo[k]fluoranthene skeleton.

As for a physical property required for a material suitable for blue-light emission in an organic EL display, it is important that a luminescent material have an emission peak in the range of 430 to 480 nm.

When an organic compound used as a material for an organic electroluminescent device (organic EL device) has a molecular weight of 1,000 or less, sublimation purification is effectively employed as a final purification method to achieve a high purity.

Furthermore, in preparation of an organic EL device, vapor deposition or application is mainly used.

When sublimation purification or vapor deposition is employed, the material is treated in a high vacuum at a pressure of about 10⁻³ Pa at a temperature of 300° C. or higher. In such a case, if the material has low thermal stability, decomposition or a reaction occurs and physical properties derived from the original material cannot be obtained.

When an organic compound of a dimer having a benzo[k]fluoranthene skeleton as a basic skeleton is used in a light-emitting device, examples of a method of controlling the emission wavelength include two methods, namely, a method of changing a bonding position of the dimer and a method of introducing a substituent. In such a case, a dimer in which the skeletons are bonded at the same position has an electronic state of a π-π* state. This is because the skeletons are bonded at moieties where the electronic levels of the benzo[k]fluoranthene skeletons are in the same state. On the other hand, when the skeletons are bonded at positions different from each other, the skeletons are bonded at moieties where the electronic states of the benzo[k]fluoranthene skeletons are different from each other. As a result, a charge transfer (CT) interaction is relatively increased. Accordingly, the emission wavelength can be increased to about 450 nm without introducing a substituent. That is, the wavelength of blue light can be controlled with a stable molecular structure as compared with a case where a substituent is introduced.

Among organic compounds of dimers having a benzo[k]fluoranthene skeleton as a basic skeleton, a dimer may be formed by bonding the 3-position of a benzo[k]fluoranthene skeleton to the 3-position of another benzo[k]fluoranthene skeleton. In this case, the 4-position of fluoranthene or benzo[k]fluoranthene has a very high reactivity compared with normal naphthalene, and thus readily causes a cyclization reaction by heat. Specifically, a compound represented by a formula below, i.e., a dimer in which the 3-position of a benzo[k]fluoranthene skeleton is bonded to the 3-position of another benzo[k]fluoranthene skeleton causes a cyclization reaction. Note that the compound represented by the formula below, which is a dimer formed by bonding the 3-position of a benzo[k]fluoranthene skeleton to the 3-position of another benzo[k]fluoranthene skeleton has a tail-to-tail bond.

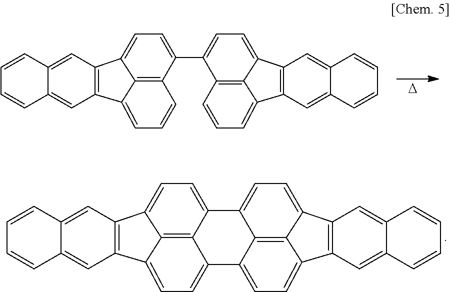

This suggests that when the above compound is used as a material of an organic EL device, the compound may be reacted by heat applied during sublimation purification, vapor deposition, or driving of the device. If the above cyclization reaction occurs, the absorption and emission wavelengths of the compound are significantly shifted to the long-wavelength side. This phenomenon causes a problem that light emission occurs at a wavelength range different from that of the original compound and light emission of the original compound is absorbed by the cyclized compound, thereby decreasing the emission intensity.

This is very important in terms of molecular design when a benzo[k]fluoranthene skeleton is used in a light-emitting device. According to organic compounds used in the present invention, benzo[k]fluoranthene skeletons are bonded at positions different from each other, and thus the compounds do not have a moiety that is cyclized by heat. This structure can suppress a chemical reaction caused by heat applied during sublimation purification, vapor deposition, and driving of the device.

Furthermore, benzo[k]fluoranthene has high planarity, and thus unsubstituted benzo[k]fluoranthene readily forms an excimer. Therefore, by introducing phenyl groups to the 7-position and the 12-position, which are located near the center of the skeleton, the phenyl groups are disposed substantially orthogonal to the benzo[k]fluoranthene skeleton. This structure is effective for suppressing the formation of an excimer. In addition, since these positions are orthogonal to the benzo[k]fluoranthene skeleton, the phenyl groups do not significantly affect the emission wavelength of benzo[k]fluoranthene.

Furthermore, the wavelength range of blue corresponds to 430 to 480 nm, and it is necessary to obtain a wavelength in the range of about 440 to 480 nm in order to realize a blue color having higher color purity. For this purpose, the emission wavelength can be further increased by introducing a substituent. However, introduction of substituents increases instability of the molecule and thus, it is desirable that substituents are not introduced.

The position to which a substituent is introduced is not particularly limited. However, a substituent is preferably introduced to the 2-position to the 5-position of benzo[k]fluoranthene, the positions being effective for increasing the emission wavelength. Furthermore, a substituent is more preferably introduced to the 3-position or the 4-position, which has a high reactivity.

7,12-Diphenylbenzo[k]fluoranthene which is a raw material of the organic compound represented by general formula (I) can be synthesized by a synthetic route 1 or 2 shown below with reference to Journal of Organic Chemistry (1952), 17, 845-54 or Journal of the American Chemical Society (1952). Furthermore, by introducing bromine to any of R₁₁ to R₁₅, 7,12-diphenylbenzo[k]fluoranthene skeletons can be bonded to each other.

As for substituents, 7,12-diphenylbenzo[k]fluoranthene in which hydrogen atoms are substituted with other substituents such as an alkyl group, a halogen atom, and a phenyl group can be similarly synthesized.

### Synthetic Route 1

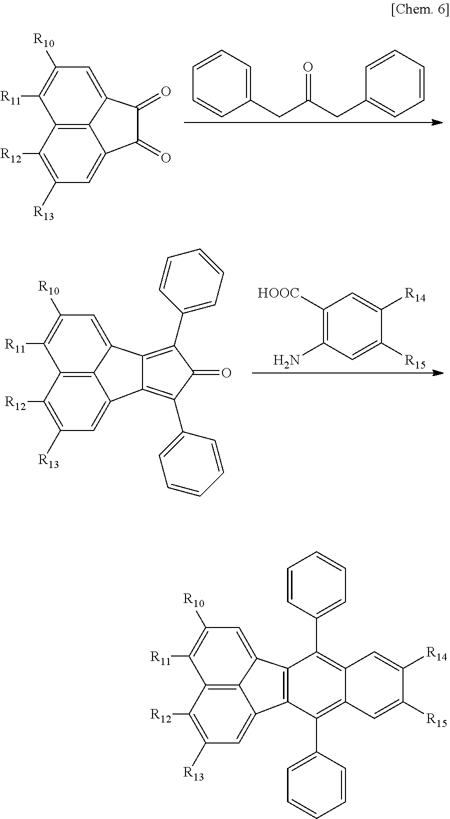

### Synthetic Route 2

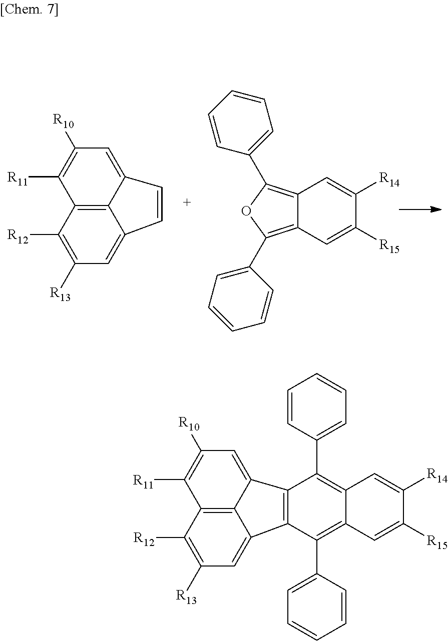

Next, an organic light-emitting device according to the present invention will now be described.

The organic light-emitting device according to the present invention includes at least a pair of electrodes, i.e., an anode and a cathode, and an organic compound layer disposed between the electrodes. This organic compound layer contains the organic compound represented by general formula (I) above. An organic light-emitting device is an device in which a luminescent material, which is an organic compound, disposed between the pair of electrodes emits light.

When one layer constituting the organic compound layer is a light-emitting layer, the light-emitting layer may be composed of only the organic compound according to the present invention or may partly contain the organic compound according to the present invention.

The phrase “light-emitting layer may partly contain the organic compound according to the present invention” means that the organic compound according to the present invention may be a main component of the light-emitting layer or an auxiliary component thereof.

Herein, among all compounds constituting the light-emitting layer, the term “main component” refers to a compound contained in a large amount in terms of weight or the number of moles, and the term “auxiliary component” refers to a compound contained in a small amount.

A material used as the main component can also be referred to as “host material”.

A material used as the auxiliary component can also be referred to as “dopant (guest) material”, “luminescence assist material” or “charge injection material”.

In the above-described cases, when the above compound is used as the light-emitting layer, the compound can be used alone as the light-emitting layer, or as a dopant (guest) material, a luminescence assist material, a host material, or a charge injection material. When a fused aromatic compound of the present invention is used as a dopant material in the organic light-emitting device of the present invention, the dopant concentration relative to a host material is preferably in the range of 0.01 to 20 percent by weight, and more preferably, in the range of 0.5 to 10 percent by weight. In addition, by controlling the concentration, the emission wavelength can be increased by about 5 to 20 nm relative to the wavelength of a solution.

When the light-emitting layer is composed of a host material having a carrier transport property and a guest material, a main process leading to light emission includes the following steps.

1. Transport of electrons and holes in the light-emitting layer

2. Generation of excitons of the host material

3. Transmission of excitation energy between molecules of the host material

4. Transfer of the excitation energy from the host material to the guest material

A desired energy transfer and light emission in each of the steps occur in competition with various deactivation steps.

In order to increase the luminous efficiency of an organic light-emitting device, it is obvious that the emission quantum yield of a luminescence center material is increased. However, how the energy transfer between a host material and a host material or between a host material and a guest material is efficiently performed is also an important factor. Furthermore, the cause of luminescence degradation due to energization has not yet become clear, but it is assumed that such degradation relates to at least an environmental change in a luminescence center material itself or a luminescent material due to peripheral molecules of the luminescence center material.

Under these circumstances, the inventors of the present invention conducted various studies and found that an device in which the compound of the present invention represented by general formula (I) is used as a host material or a guest material of a light-emitting layer, in particular, as a guest material thereof has an optical output with a high efficiency and a high luminance and has a very high durability.

Next, an organic light-emitting device of the present invention will be described.

The organic light-emitting device according to the present invention includes a cathode, an anode, and an organic compound layer disposed between the anode and the cathode, wherein the organic compound layer contains an organic compound in which two 7,12-diphenylbenzo[k]fluoranthene skeletons each of which may have a substituent are bonded head-to-tail or bonded tail-to-tail at positions different from each other.

The organic compound layer may contain the organic compound represented by general formula (I).

The organic compound layer may be a light-emitting layer.

Furthermore, an image display apparatus including this organic light-emitting device and a unit arranged to supply the organic light-emitting device with an electrical signal can be provided.

In the organic light-emitting device according to the present invention, it is sufficient that the organic compound layer disposed between the anode and the cathode contains at least an organic compound in which two 7,12-diphenylbenzo[k]fluoranthene skeletons each of which may have a substituent are bonded head-to-tail or bonded tail-to-tail at positions different from each other. In such a case, the organic compound layer may be composed of only the organic compound or may at least contain a small amount of the organic compound. Alternatively, the organic compound layer may contain various types of the organic compound.

The organic light-emitting device according to the present invention may include only this organic compound layer or include at least one other layer. In this case, the organic light-emitting device is a multilayer organic light-emitting device.

A first example to a fifth example of such a multilayer organic light-emitting device will be described below.

The first example of the multilayer organic light-emitting device has a structure in which an anode, a light-emitting layer, and a cathode are sequentially provided on a substrate. The light-emitting layer used in this example is useful in the case where the light-emitting layer has a hole-transport performance, an electron-transport performance, and a light-emitting performance by itself or the case where compounds having these characteristics are used as a mixture.

The second example of the multilayer organic light-emitting device has a structure in which an anode, a hole-transporting layer, an electron-transporting layer, and a cathode are sequentially provided on a substrate. This structure is useful in the case where a material having either a hole-transporting property or an electron-transporting property, or both the hole-transporting property and the electron-transporting property is used as each of the layers, and a luminescent substance is used in combination with a simple hole-transporting substance or electron-transporting substance that does not have a light-emitting property. In this case, a light-emitting layer is composed of either the hole-transporting layer or the electron-transporting layer.

The third example of the multilayer organic light-emitting device has a structure in which an anode, a hole-transporting layer, a light-emitting layer, an electron-transporting layer, and a cathode are sequentially provided on a substrate. This is an device in which functions of carrier transportation and light emission are separated from each other. A luminescent substance can be used in combination with compounds having a hole-transporting property, an electron-transporting property, and a light-emitting property as required. In addition, the degree of freedom of material selection is significantly increased, and various compounds having different emission wavelengths can be used. Consequently, the hue of light emission can be diversified. Furthermore, carriers or excitons are effectively confined in the light-emitting layer disposed at the center, thereby improving the luminous efficiency.

The fourth example of the multilayer organic light-emitting device has a structure in which an anode, a hole injection layer, a hole-transporting layer, a light-emitting layer, an electron-transporting layer, and a cathode are sequentially provided on a substrate. This structure is advantageous in that the adhesion between the anode and the hole-transporting layer is improved and a hole injection property is improved. Accordingly, this structure is effective to realize a reduction in the voltage.

The fifth example of the multilayer organic light-emitting device has a structure in which an anode, a hole injection layer, a hole-transporting layer, a light-emitting layer, a hole/exciton-blocking layer, an electron-transporting layer, and a cathode are sequentially provided on a substrate. This is a structure in which a layer (hole/exciton-blocking layer) that blocks a hole or an exciton from passing through the cathode side is interposed between the light-emitting layer and the electron-transporting layer. According to this structure, the luminous efficiency can be effectively improved by using a compound having a very high ionization potential as the hole/exciton-blocking layer.

A light emission region is a region where the organic compound according to the present invention is present. The organic compound according to the present invention is an organic compound in which two 7,12-diphenylbenzo[k]fluoranthene skeletons each of which may have a substituent are bonded head-to-tail or bonded tail-to-tail at positions different from each other. More preferably, the light emission region is a region that contains the organic compound represented by general formula (I). In the above-described examples, a region of the light-emitting layer corresponds to the light emission region.

However, the first example to the fifth example of the multilayer organic light-emitting device are merely very basic device structures, and the structure of an organic light-emitting device including the organic compound according to the present invention is not limited to the above examples.

For example, an insulating layer, an adhesive layer, or an interference layer may be provided between an electrode and an organic layer. Alternatively, the electron-transporting layer or the hole-transporting layer may be composed of two layers having different ionization potentials. Thus, the organic light-emitting device may have various layer structures.

The compound represented by general formula (I) used in the present invention can be used in any of the first example to the fifth example described above.

In the organic light-emitting device according to the present invention, a layer containing an organic compound contains at least one compound represented by general formula (I) used in the present invention, and the compound represented by general formula (I) is particularly used as a guest material of a light-emitting layer.

In addition to the organic compound according to the present invention, a known low-molecular weight or high-molecular weight hole-transporting compound, luminescent compound, electron-transporting compound, or the like may be used in combination as required.

Examples of the compounds will be described below.

As hole injection/transport materials, materials having a high hole mobility are preferably used so that holes can be easily injected from an anode and the injected holes are transported to a light-emitting layer. Examples of the low-molecular weight and high-molecular weight materials having a hole injection/transport performance include, but are not limited to, triarylamine derivatives, phenylenediamine derivatives, stilbene derivatives, phthalocyanine derivatives, porphyrin derivatives, poly(vinylcarbazole), poly(thiophene), and other electrically conductive polymers.

Examples of host materials mainly include, but are not limited to, not only the compounds shown in Table 1 and derivatives of the compounds shown in Table 1, but also fused ring compounds (such as fluorene derivatives, naphthalene derivatives, anthracene derivatives, pyrene derivatives, carbazole derivatives, quinoxaline derivatives, and quinoline derivatives), organoaluminum complexes such as tris(8-quinolinolato) aluminum, organozinc complexes, triphenylamine derivatives, and polymer derivatives such as poly(fluorene) derivatives and poly(phenylene) derivatives.

TABLE 1
H1

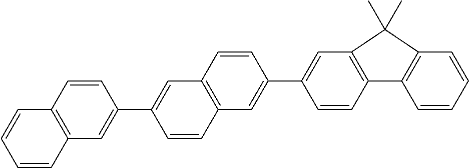

H2

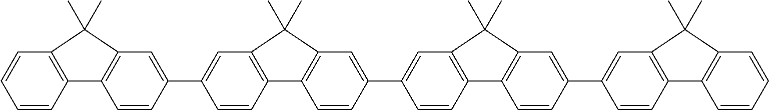

H3

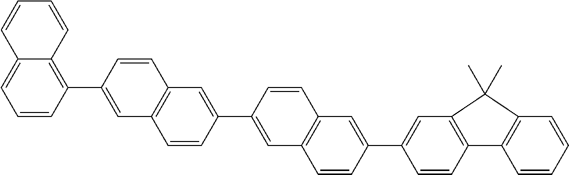

H4

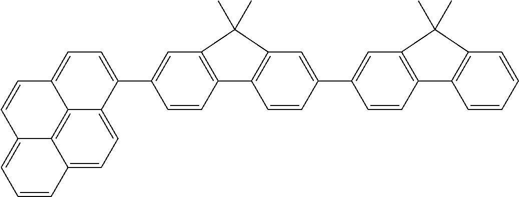

H5

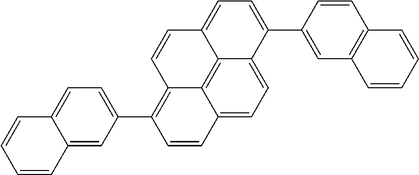

H6

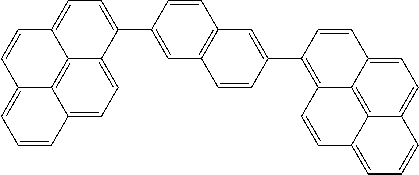

H7

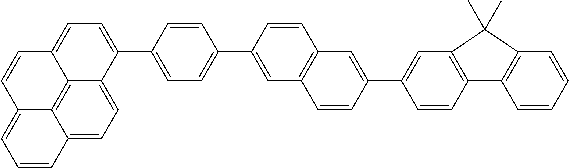

H8

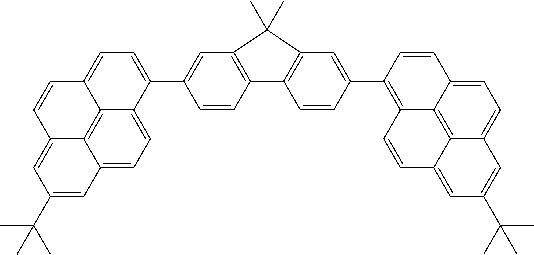

H9

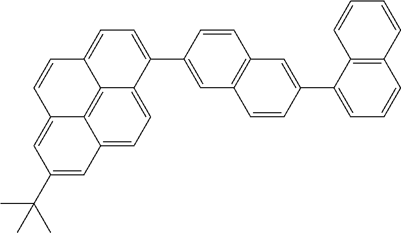

H10

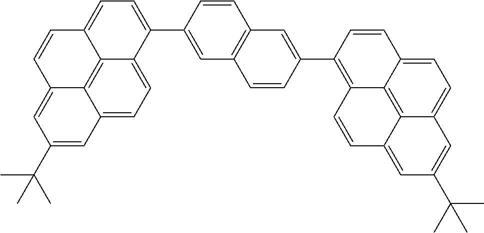

H11

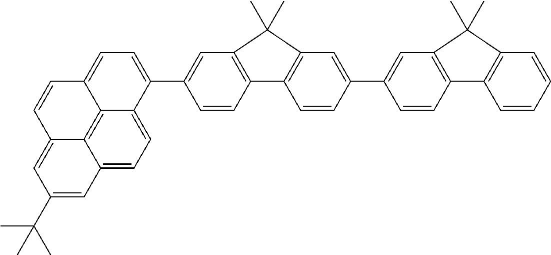

H12

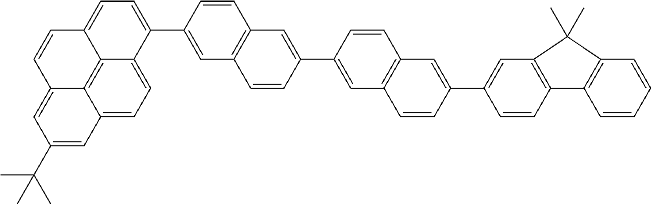

H13

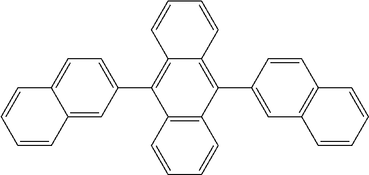

H14

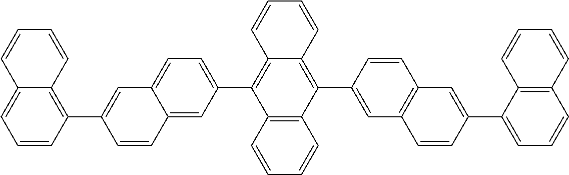

H15

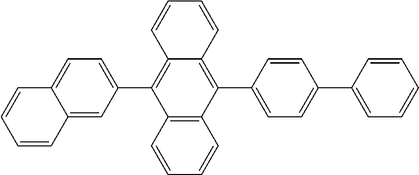

H16

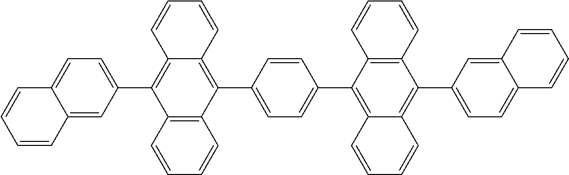

H17

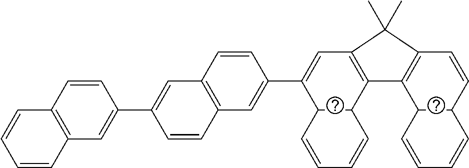

H18

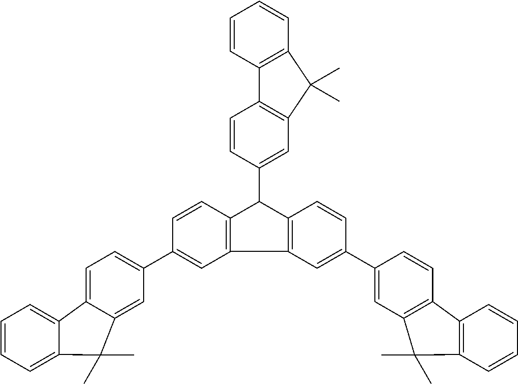

H19

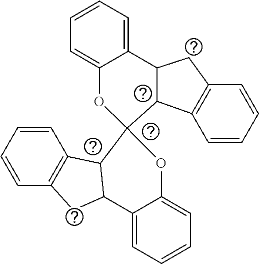

H20

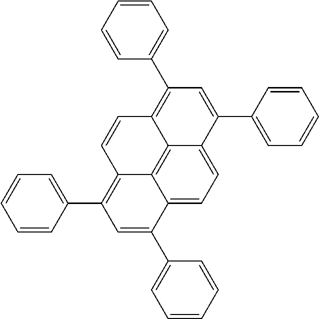

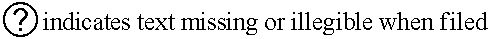

Electron injection/transport materials can be selected from materials to which electrons are easily injected from a cathode and which can transport the injected electrons to the light-emitting layer and in consideration of, for example, the balance with the hole mobility of the hole injection/transport material. Examples of the materials having an electron injection/transport performance include, but are not limited to, oxadiazole derivatives, oxazole derivatives, pyrazine derivatives, triazole derivatives, triazine derivatives, quinoline derivatives, quinoxaline derivatives, phenanthroline derivatives, and organoaluminum complexes.

As anode materials, those having a work function as high as possible are preferable. Examples of the anode materials that can be used include metal devices such as gold, platinum, silver, copper, nickel, palladium, cobalt, selenium, vanadium, and tungsten; alloys thereof; and metal oxides such as tin oxide, zinc oxide, indium oxide, indium tin oxide (ITO), and indium zinc oxide. In addition, electrically conductive polymers such as polyaniline, polypyrrole, and polythiophene can also be used. These electrode materials may be used alone or in combinations of two or more materials. The anode may be composed of one layer or two or more layers.

On the other hand, as cathode materials, those having a low work function are preferable. Examples of the cathode materials include metal devices such as alkali metals, e.g., lithium; alkaline earth metals, e.g., calcium; aluminum; titanium; manganese; silver; lead; and chromium. Alloys combining these metal devices can also be used. For example, magnesium-silver, aluminum-lithium, aluminum-magnesium, or the like can be used. Metal oxides such as indium tin oxide (ITO) can also be used. These electrode materials may be used as alone or in combinations of two or more materials. The cathode may be composed of one layer or two or more layers.

Examples of the substrate used in the organic light-emitting device of the present invention include, but are not particularly limited to, opaque substrates such as metal substrates and ceramic substrates, and transparent substrates such as glass, quartz, and plastic sheets. Alternatively, the luminescent color can be controlled by providing a color filter film, a fluorescent color conversion filter film, a dielectric reflecting film, or the like on the substrate.

In addition, a protective layer or a sealing layer may be provided on a prepared device for the purpose of preventing contact with to oxygen, moisture, or the like. Examples of the protective layer include a diamond thin film; inorganic material films such as metal oxide films and metal nitride films; polymer films such as fluorocarbon resin films, a polyethylene film, silicone resin films, and a polystyrene film; and photocurable resin films. The device may be covered with, for example, glass, a gas-impermeable film, or a metal and packaged with a suitable sealing resin.

In the organic light-emitting device of the present invention, a layer containing the organic compound of the present invention and layers composed of the other organic compounds are formed by the methods described below. In general, a thin film is formed by a vacuum evaporation method, an ionized vapor deposition method, a sputtering method, a method using plasma, or a known coating method (for example, spin coating, dipping, a cast method, an LB method, or an ink jet method) after a material is dissolved in a suitable solvent. Among these, when a layer is formed by a vacuum evaporation method or a solution coating method, crystallization does not readily occur and thus the resulting layer has good stability with time. When a film is formed by a coating method, the material may be combined with a suitable binder resin to form the film.

Examples of the binder resin include, but are not limited to, polyvinylcarbazole resins, polycarbonate resins, polyester resins, ABS resins, acrylic resins, polyimide resins, phenolic resins, epoxy resins, silicone resins, and urea resins. These binder resins may be used alone as a homopolymer or a copolymer or used as a mixture of two or more types of resin. Furthermore, additives such as a known plasticizer, antioxidant, or ultraviolet absorbent may be optionally used in combination.

The organic light-emitting device of the present invention can be applied to a product which needs energy saving and a high luminance. Application examples thereof include display apparatuses, illuminating devices, light sources of a printer, and backlights of a liquid crystal display apparatus.

As a display apparatus, a lightweight, flat-panel display that has a high visibility and that realizes energy saving can be obtained. The display apparatus can be used as an image display apparatus such as a PC, a television, or an advertizing medium. Alternatively, the display apparatus may be used in a display unit of an image pickup apparatus such as a digital still camera or a digital video camera.

Alternatively, the display apparatus may be used in an operation display unit of an electrophotographic image-forming apparatus, namely, a laser beam printer, a copy machine, or the like.

Alternatively, the display apparatus can be used as a light source that is used when a latent image is exposed on a photosensitive member of an electrophotographic image-forming apparatus, namely, a laser beam printer, a copy machine, or the like. A plurality of organic light-emitting devices that can be independently addressed are arranged in the form of an array (for example, in the form of a line) and a desired exposure is performed on a photosensitive drum, thereby forming a latent image. The use of organic light-emitting devices of the present invention can decrease a space that has been required for arranging a light source, a polygon mirror, and various optical lenses to date.

As for the illuminating devices and the backlights, an energy-saving effect obtained by the present invention can be expected. The organic light-emitting devices of the present invention can be used as a surface light source.

As described above, the luminescent color can be controlled by providing a color filter film, a fluorescent color conversion filter film, a dielectric reflecting film, or the like on a substrate supporting the organic light-emitting device of the present invention. A thin-film transistor (TFT) may be provided on the substrate and the organic light-emitting device may be connected to the TFT, thereby the emission/non-emission can be controlled. Either a source electrode or a drain electrode of the TFT is connected to either the anode or the cathode of the organic light-emitting device. Alternatively, a plurality of organic light-emitting devices may be arranged in a matrix shape, that is, arranged in an in-plane direction and used as an illuminating device.

Next, a display apparatus including an organic light-emitting device of the present invention will be described. This display apparatus includes organic light-emitting devices of the present invention and TFTs that control the light-emission luminance of the organic light-emitting devices. Furthermore, the display apparatus optionally includes a unit arranged to supply the organic light-emitting devices of the present invention with an electrical signal. By controlling the organic light-emitting devices by the TFTs, an active matrix display apparatus can be provided.

FIGURE is a schematic cross-sectional view of a display apparatus including organic light-emitting devices in a pixel portion. The FIGURE shows two organic light-emitting devices and two TFTs. One organic light-emitting device is connected to one TFT. As shown in the FIGURE, a display apparatus 3 includes a substrate 31 composed of, for example, glass and a moisture-proof film 32 for protecting components (TFT and an organic layer) formed on an upper portion thereof. As a material constituting the moisture-proof film 32, silicon oxide, a composite material of silicon oxide and silicon nitride, or the like is used. A gate electrode 33 is provided on the moisture-proof film 32. The gate electrode 33 is formed by depositing a metal such as chromium (Cr) by sputtering.

A gate insulating film 34 is arranged so as to cover the gate electrode 33. The gate insulating film 34 is formed by depositing, for example, silicon oxide by a plasma vapor deposition (CVD) method, a catalytic chemical vapor deposition (cat-CVD) method, or the like, and pattering the deposited film. A semiconductor layer 35 is provided so as to cover the gate insulating film 34 disposed in each patterned region to be formed into a TFT. This semiconductor layer 35 is formed by depositing a silicon film by a plasma CVD method or the like (and annealing the film at a temperature of 290° C. or higher in some cases), and patterning the silicon film in accordance with a circuit shape.

Furthermore, a drain electrode 36 and a source electrode 37 are provided on each semiconductor layer 35. Thus, each TFT device 38 includes the gate electrode 33, the gate insulating film 34, the semiconductor layer 35, the drain electrode 36, and the source electrode 37. An insulating film 39 is provided on the TFT devices 38. A contact hole (through-hole) 310 is provided in the insulating film 39. An anode 311 for an organic light-emitting device, the anode being composed of a metal, a metal oxide, or the like, is connected to the source electrode 37 via the contact hole 310.

On the anode 311, a multilayered or single-layered organic layer 312 and a cathode 313 are sequentially stacked, thus constituting the organic light-emitting device. In this embodiment, in order to prevent degradation of the organic light-emitting device, a first protective layer 314 or a second protective layer 315 may be provided on the organic light-emitting device.

In the above display apparatus, the switching device is not particularly limited. In addition to the TFT described above, a single-crystal silicon substrate, an MIM device, an amorphous-Si (a-Si) type device, or the like can also be easily used.

On the ITO electrode, a multilayered or single-layered organic light-emitting layer and a cathode layer are sequentially stacked. Thus, an organic light-emitting display panel can be obtained. By driving the display panel including the organic compound of the present invention, a display that has a good image quality and that is stable for a long time can be realized.

As for a direction of output of the light from the device, either one of the bottom emission configuration (configuration in which light is output from the substrate side) or the top emission configuration (configuration in which light is output from the side opposite the substrate) may be used.

### EXAMPLES

The present invention will be more specifically described on the basis of Examples, but the present invention is not limited thereto.

### Example 1

### Synthesis of Exemplified Compound A1

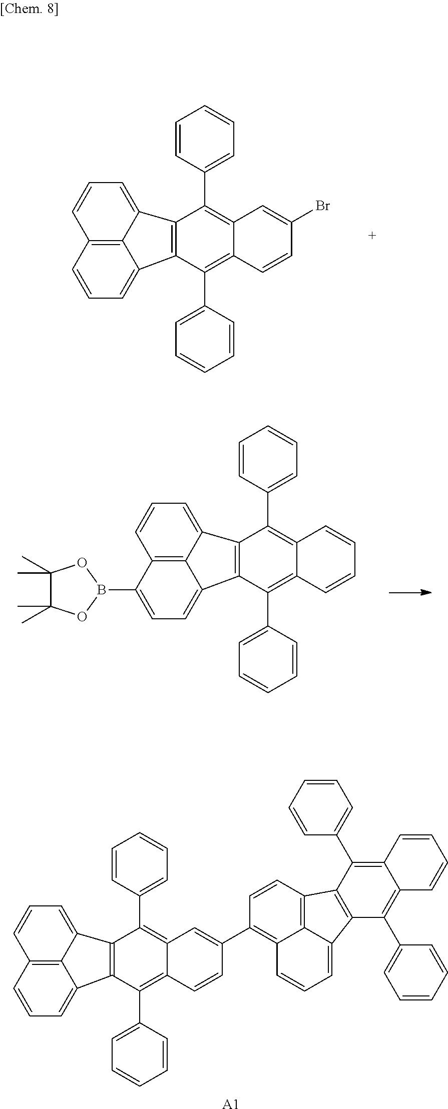

First, 966 mg (2 mmole) of 9-bromo-7,12-diphenylbenzo[k]fluoranthene, 1,060 mg (2 mmole) of 2-(fluoranthene-3-yl)-4,4,5,5-tetramethyl-1,3,2-dioxa borane, 0.05 g of Pd(PPh₃)₄, 20 mL of toluene, 10 mL of ethanol, and 20 mL of a 2M-aqueous sodium carbonate solution were charged in a 100-mL round bottom flask, and the mixture was stirred under nitrogen at 80° C. for eight hours. After the reaction, the resulting crystals were separated by filtration, and dispersed and washed in water, ethanol, and heptane. The crystals were dissolved in toluene under heating, and the solution was subjected to hot filtration. The resulting crystals were recrystallized with toluene. The crystals were dried in a vacuum at 120° C., and purified by sublimation. Thus, 1,260 mg of pale yellow crystals of Compound A1 were obtained (yield: 780).

The structure of this compound was confirmed by an NMR measurement.

¹H NMR (CDCl₃, 500 MHz) σ (ppm): 7.80-7.52 (m, 34H), 7.40-7.38 (m, 1H), 7.35-7.28 (m, 1H), 6.65-6.63 (t, 1H), 6.61-6.59 (m, 1H).

An emission spectrum of a toluene solution of Exemplified Compound A1 with a concentration of 1×10⁻⁵ mol/L was measured using a fluorescence spectrophotometer F-4500 manufactured by Hitachi, Ltd. at an excitation wavelength of 350 nm. According to the measurement result of photoluminescence, the spectrum had a maximum intensity at 449 nm.

### Comparative Example 1

A comparison of thermal stability was performed using

Compound E1 as a comparative example relative to the idea of the present invention.

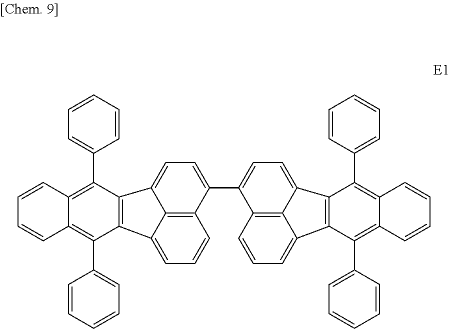

A material of Compound B1 used in a light-emitting device of the present invention and a material of Compound E1 used as the comparative example were heated to 360° C. in a vacuum at a pressure of 2.0×10⁻¹ Pa. Consequently, the color of Compound E1 gradually turned to red, and an emission peak due to Compound E2 could be confirmed. Although Compound B1 was melted and the color thereof turned to yellow, another compound was not confirmed in an analysis after cooling.

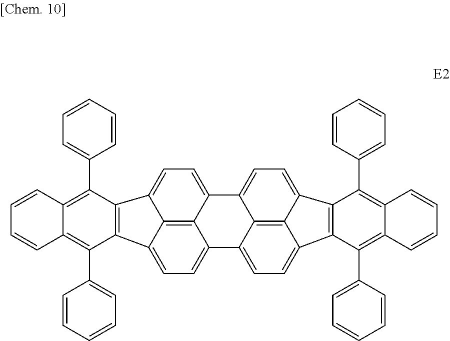

### Comparative Example 2

An analysis of the emission wavelength was conducted using Compound E3 as a comparative example relative to the idea of the present invention.

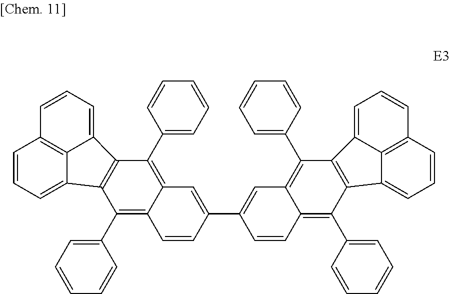

An emission spectrum of a toluene solution of Compound E3 with a concentration of 1×10⁻⁵ mol/L was measured using a fluorescence spectrophotometer F-4500 manufactured by Hitachi, Ltd. at an excitation wavelength of 350 nm. According to the measurement result of photoluminescence, the spectrum had a maximum intensity at 437 nm. On the other hand, the emission wavelength of Exemplified Compound A1 was 449 nm. Thus, the emission wavelength could be shifted to the long-wavelength side on the basis of the idea of the present invention without introducing a substituent.

### Examples 2 to 10

In each of these Examples, the device described in the fifth example of the multilayer organic light-emitting device (anode/hole injection layer/hole-transporting layer/light-emitting layer/hole•exciton-blocking layer/electron-transporting layer/cathode) was prepared. First, an ITO film having a thickness of 100 nm was patterned on a glass substrate. On the substrate having the ITO film thereon, the following organic layers and an electrode layer were successively deposited by a resistance-heating vacuum evaporation method in a vacuum chamber at a pressure of 10⁻³ Pa so that an area of the facing electrodes was 3 mm².

Hole-transporting layer (30 nm): F-1

Light-emitting layer (30 nm); Host material: F-2, Guest material: Exemplified Compound (weight ratio 5%)

Hole/exciton-blocking layer (10 nm): F-3

Electron-transporting layer (30 nm): F-4

Metal electrode layer 1 (1 nm): LiF

Metal electrode layer 2 (100 nm): A1

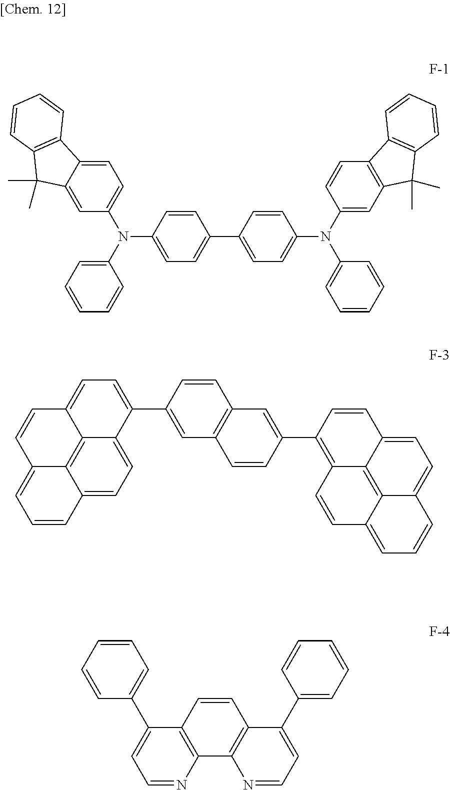

Current-voltage characteristics of each EL device were measured with a microammeter 4140B manufactured by Hewlett-Packard Development Company, and the light-emission luminance thereof was measured with a luminance meter BM7 manufactured by Topcon Corporation.

The luminous efficiency and the voltage of Example 2 to Example 10 are shown in Table 2.

TABLE 2

| | | | Luminous | |
|---|---|---|---|---|
| | | | Efficiency | Voltage |
| | Guest | F-2 | (cd/A) | (V) |
| Example 2 | A1 | H4 | 4.2 | 3.7 |
| Example 3 | A1 | H11 | 5.0 | 3.8 |
| Example 4 | A1 | H12 | 4.7 | 3.8 |
| Example 5 | A2 | H5 | 4.8 | 3.8 |
| Example 6 | A3 | H12 | 5.0 | 3.7 |
| Example 7 | A3 | H15 | 4.2 | 4.0 |
| Example 8 | A15 | H4 | 4.7 | 3.8 |
| Example 9 | B1 | H2 | 4.2 | 4.2 |
| Example 10 | B1 | H18 | 3.8 | 4.0 |

### Results and Discussion

According to the organic compounds of the present invention, two 7,12-diphenylbenzo[k]fluoranthene skeletons are bonded head-to-tail or bonded tail-to tail at positions different from each other. Consequently, unlike a compound having a tail-to-tail bond at the same position, the organic compounds of the present invention were not chemically reacted by heat. Thus, more suitable compounds that emit blue light could be obtained without introducing a substituent to a compound having a head-to-head bond. In addition, good emission characteristics could be obtained by using this material in a light-emitting device.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2008-295801, filed Nov. 19, 2008, which is hereby incorporated by reference herein in its entirety.

1. An organic light-emitting device comprising:
a cathode;
an anode; and
an organic compound layer disposed between the anode and the cathode,
wherein the organic compound layer contains an organic compound in which two 7,12-diphenylbenzo[k]fluoranthene skeletons each of which may have a substituent are bonded head-to-tail or bonded tail-to-tail at positions different from each other.

2. The organic light-emitting device according to claim 1, wherein the organic compound is represented by general formula (1): 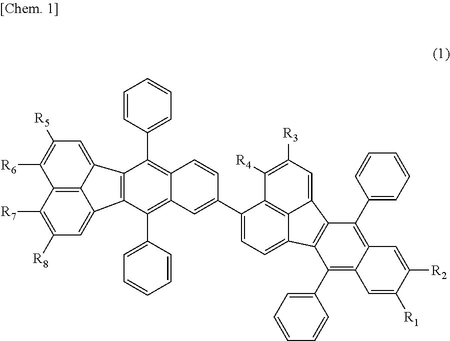
wherein R₁ to R₈ are each independently selected from a hydrogen atom, a halogen atom, a substituted or unsubstituted alkyl group, a substituted or unsubstituted alkoxy group, a substituted or unsubstituted aralkyl group, a substituted or unsubstituted amino group, a substituted or unsubstituted aryl group, and a substituted or unsubstituted heterocyclic group.

3. The organic light-emitting device according to claim 1, wherein the organic compound layer is a light-emitting layer.

4. An image display apparatus comprising:
the organic light-emitting device according to claim 1; and
a unit arranged to supply the organic light-emitting device with an electrical signal.

5. An organic compound represented by general formula (I): 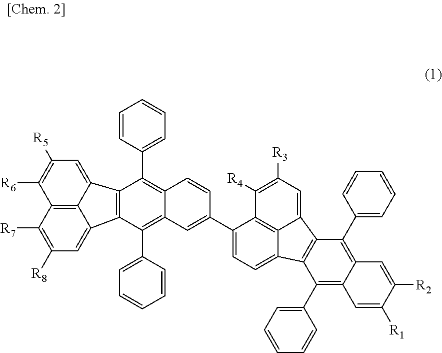
wherein R₁ to R₈ are each independently selected from a hydrogen atom, a halogen atom, a substituted or unsubstituted alkyl group, a substituted or unsubstituted alkoxy group, a substituted or unsubstituted aralkyl group, a substituted or unsubstituted amino group, a substituted or unsubstituted aryl group, and a substituted or unsubstituted heterocyclic group.
